(19) United States
(12) Patent Application Publication
(10) Pub. No.: US 2003/0100881 A1
(43) Pub. Date: May 29, 2003

(54) Collapsible needle holder

(75) Inventor: Charles G. Hwang, Ridgewood, NJ (US)

Correspondence Address: Kirk M. Miles, Esq., WEBB ZIESENHEIM LOGSDON ORKIN & HANSON, P.C., 700 Koppers Building, 436 Seventh Avenue, Pittsburgh, PA, 15219-1818, US

(73) Assignee: Becton, Dickinson and Company

(21) Appl. No.: 10/299,889

(22) Filed: Nov. 19, 2002

Related U.S. Application Data
(60) Provisional application No. 60/333,475, filed on Nov. 28, 2001.

(51) Int. Cl. A61B 19/00

(52) U.S. Cl. 604/403

(57) ABSTRACT

A needle holder provides for the ease of transportation and storage. The needle holder includes an elongate body having tubular wall and opposed ends. The tubular wall is radially expandable from a flat configuration to an expanded configuration defining openings at the ends. A flap extends from one of the ends and is positionable across the opening at the one end to close the opening. The flap is capable of supporting a needle assembly thereat. A needle securement member may be attached to the flap so as to support the needle assembly. In the flat configuration, plural needle holders may be stacked for ease of transportation and storage.

### CROSS-REFERENCE TO RELATED APPLICATIONS

The present application claims priority to U.S. Provisional Application No. 60/333,475 filed on Nov. 28, 2001.

### BACKGROUND OF THE INVENTION

1. Field of the Invention

The present invention relates generally to a needle holder for supporting a specimen collection tube and, more particularly, the present invention relates to a collapsible needle holder which provides for convenient packaging and shipping.

2. Description of Related Art

Blood samples and other medical specimens are routinely taken and collected in a specimen collection container. In blood collection applications, the collection container is typically a hollow blood collection tube with one end closed by a semi-spherical portion and the other end open. The open end is sealable by an impervious elastomeric cover. The tube thus defines an interior chamber for collecting and holding the blood sample.

To collect a blood sample, the tube is used in concert with a tube holding device. This device typically comprises a hollow needle extending through the closed end of a tubular housing. The tubular housing has an opposed open end for accepting the blood collection tube. The hollow needle is inserted through the dermal layer into the lumen of the blood vessel and taps into the circulation system of a subject to direct blood therethrough towards the interior compartment of the holding device. The collection container is inserted through the open end of the holder so that the hollow needle punctures through the cover of the collection container. The interior of the collection container is now in direct communication with the circulation system and, having typically been formed in a vacuum, draws blood through the hollow needle and into the collection container. Once the phlebotomist has drawn enough blood into the collection container, the container may be removed from the holder. The container cover, being of an elastomeric material, reseals the hole made by the needle. The phlebotomist may then, if desired, insert a new collection container into the holder to draw more blood.

As may be appreciated, the use of such needle holders is widespread. Many if not all of the current needle holders are designed for “single use only” thus increasing the number of needle holders used. Furthermore, these needle holders are traditionally formed of molded plastic and, while they serve adequately for the intended purposes, they may be more costly to manufacture then would be desirable for a disposable product. In addition, the needle holders are usually shipped in bulk packages with plural needle holders being contained in a shipping carton. As the needle holders are formed in a cylindrical configuration, this method of shipment greatly increases the cost of shipping and storage of the needle holders.

It is therefore desirable to provide a needle holder which may be manufactured economically and provided in a configuration which requires less space for shipping and storage.

### SUMMARY OF THE INVENTION

The present invention provides an improved needle holder which is economical to manufacture and requires less space for shipping and storage. The needle holder of the present invention includes an elongate body having a tubular wall and opposed ends. The tubular wall is radially expandable from a flat configuration which facilitates shipping and storage to an expanded configuration which permits use. The expanded configuration defines openings at the ends of the tubular wall. A flap extends from one of the ends of the tubular wall and is positioned across the opening defined thereat to enclose the opening. The flap is capable of supporting the needle assembly thereat.

Preferably, the needle holder of the present invention includes a tab extending from the flap. The tab is lockably engageable with the tubular wall adjacent one end to secure the flap across the opening.

More preferably, the needle holder of the present invention includes a pair of finger grip flanges which are extendable from the tubular wall adjacent the opposite end thereof. The finger grip flanges provide for manual manipulation of the needle holder by the user. The finger grip flanges may be formed integrally with the tubular wall or may be provided by a separately attached flap insert.

More preferably, the needle holder of the present invention further includes a needle securement member which is attachable to the flap. The needle securement member includes a base which is attachable to the flap and a centrally located extending annular neck which supports the needle assembly therethrough. In preferred form, the body and the flap of the needle holder of the present invention are integrally formed from a low cost material, such as those selected from cardboard, paper, or plastic.

### BRIEF DESCRIPTION OF THE DRAWINGS

FIG. 1 is a perspective showing of an improved needle holder assembly of the present invention;

FIG. 2 shows in plan view, the flat blank forming the body of the needle holder assembly of FIG. 1;

FIGS. 3-5 show sequentially, the steps employed in forming the needle holder assembly of FIG. 1 from the flat blank of FIG. 2;

FIG. 6 shows a plurality of flat blanks of FIG. 2 in stacked arrangement;

FIGS. 7-9 show in perspective view, partially broken away, further embodiments of the needle holder assembly of the present invention; and

FIG. 10 is a perspective showing an additional embodiment of the needle holder assembly of the present invention.

### DETAILED DESCRIPTION OF THE PRESENT INVENTION

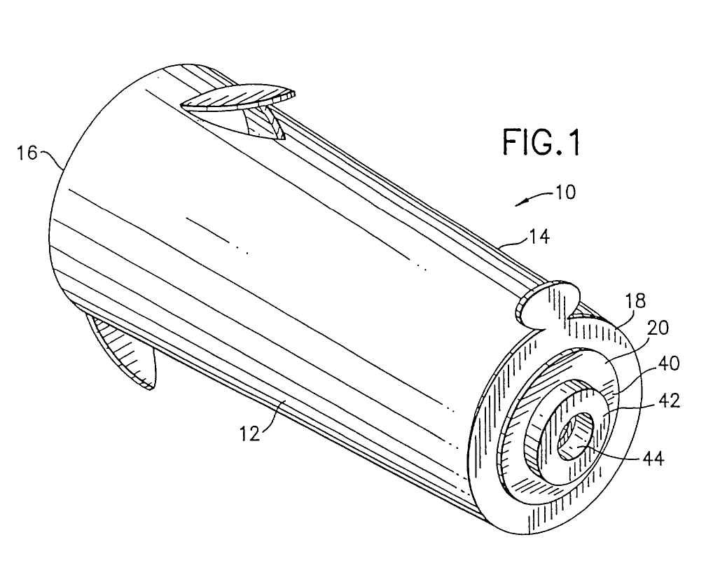

The present invention provides a needle holder which is formed from a flat collapsible stackable blank. Needle holder 10 of the present invention is shown in FIG. 1 in assembled configuration having elongate body 12. Body 12 includes a generally cylindrical wall 14 which may be slightly conically tapered from a wider open end 16 to a narrower generally closed end 18. The closed end 18 supports needle securement member 20 thereat for supporting and securing a needle assembly (not shown) therein in conventional fashion.

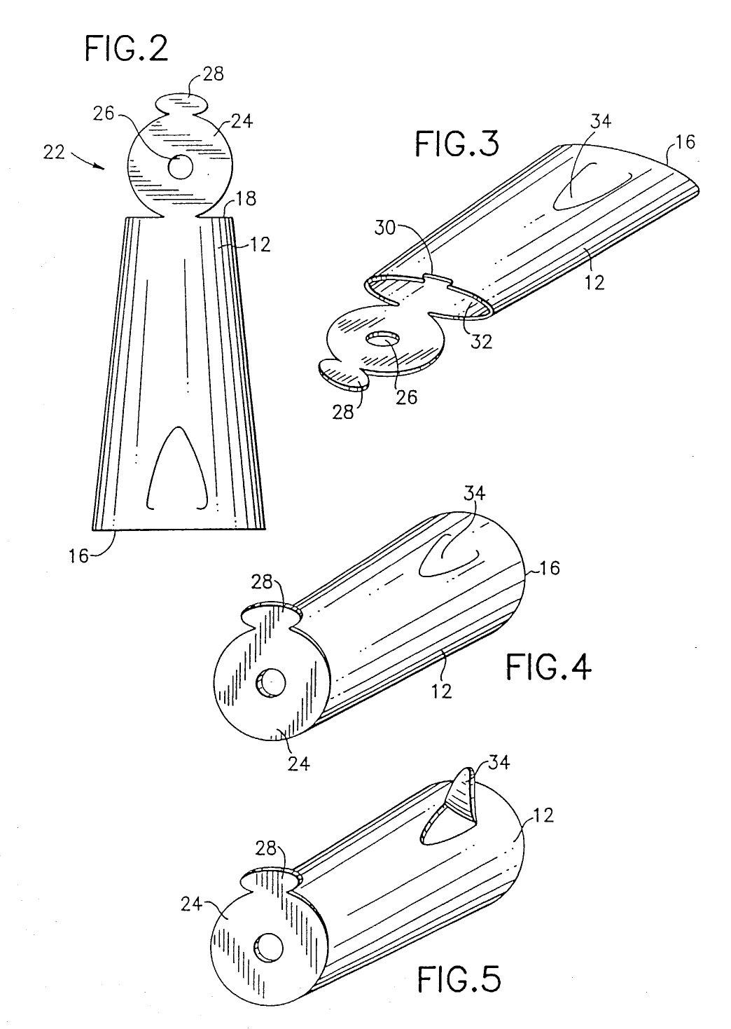

Referring to FIG. 2, the body 12 of needle holder assembly 10 is formed from a generally flat blank 22. Blank 22 may be formed from any material which is capable of radially expanding between a generally flat configuration and a tubular configuration, such as cardboard, paper, or a resilient polymeric material. Blank 22 is desirably formed from an integral die cut portion of a sheet material and arranged in the configuration shown in FIG. 2. The blank 22 defines a generally flat, two-part construction having an extending flap 24 formed adjacent an end of a tubular body 12 through a crease or fold. Flap 24 is generally circular in shape having a central circular opening 26 therethrough. Flap 24 is constructed so as to overlie and close open end 18 of body 12 when body 12 is placed in an expanded generally cylindrical tubular configuration. In that regard, flap 24 includes an extending tab 28 integrally formed therewith. Tab 28 is designed to be accommodated within a recess 30 formed in body 12 adjacent end 18. As may be appreciated, flap 24 is formed at and extends from body 12 at a location which is diametrically opposite that of slot 30. In this manner, flap 24 can be folded across end 18 closing the end with access being provided to the interior 32 of body 12 through opening 26. The blank 22 further includes a pair of diametrically opposed die cut flaps 34 adjacent opening 16. Die cut flaps 34 may be folded outwardly of the plane of body 12 as shown in FIG. 1 to form manual gripping flanges to facilitate use of the needle holder.

With reference now to FIGS. 3-5 the configuration of blank 22 from a generally flat structure into a useable needle holder assembly is shown.

As shown in FIG. 3 the blank 22 is generally flat having a flattened tubular wall in a first shape with a non-annular tubular cross-section. The blank may be grasped and expanded to form body 12 into a generally cylindrical or conical tubular configuration as a second shape with an annular tubular cross-section. In this generally cylindrical or conical tubular configuration, body 12 defines a longitudinal axis, with a pair of creases or folds extending along the length of body 12 in a manner substantially coplanar with to the axis of body 12. It is noted that body 12 is depicted in FIG. 3 with a pair of creases extending coplanar with the axis of body 12. It is contemplated, however, that other folded structures can be used to define blank 22 which can be radially expanded to form body 12. Thereafter, flap 24 may be folded over at the point at which it is joined to body 12 so that the tab 28 overlies slot 30. The tab 28 may be inserted into slot 30 to fix flap 24 across end 18. Once the flap is secured across end 18, the gripping flanges 34 may be folded out of the wall 14 as shown in FIG. 5.

The needle holder 10 of the present invention further includes a needle securement member 20 at flap 24, which needle securement member 20 extends from end 18 of body 12 when in the generally cylindrical tubular configuration. Needle securement member 20 may be integrally formed with flap 24, or may be a separate member which is separately formed and attached or affixed to flap 24 during assembly of needle holder 10. As shown in FIG. 1, needle securement member 20 may be applied to end 18 over flap 24. It is contemplated that self-stick adhesive may be employed on the undersurface of planar portion 40 to secure needle securement member 20 to flap 24.

Needle securement member 20 may be formed of molded plastic or other suitable material. Securement member 20 includes a planar portion 40 which is generally circular in shape and an extending annular neck 42. Securement member 20 includes a central aperture 44 extending through neck 42. Aperture 44 is alignable with opening 26 of flap 24. Aperture 44 may be internally screw-threaded so as to accommodate an externally threaded base of a needle (not shown) which is inserted therein.

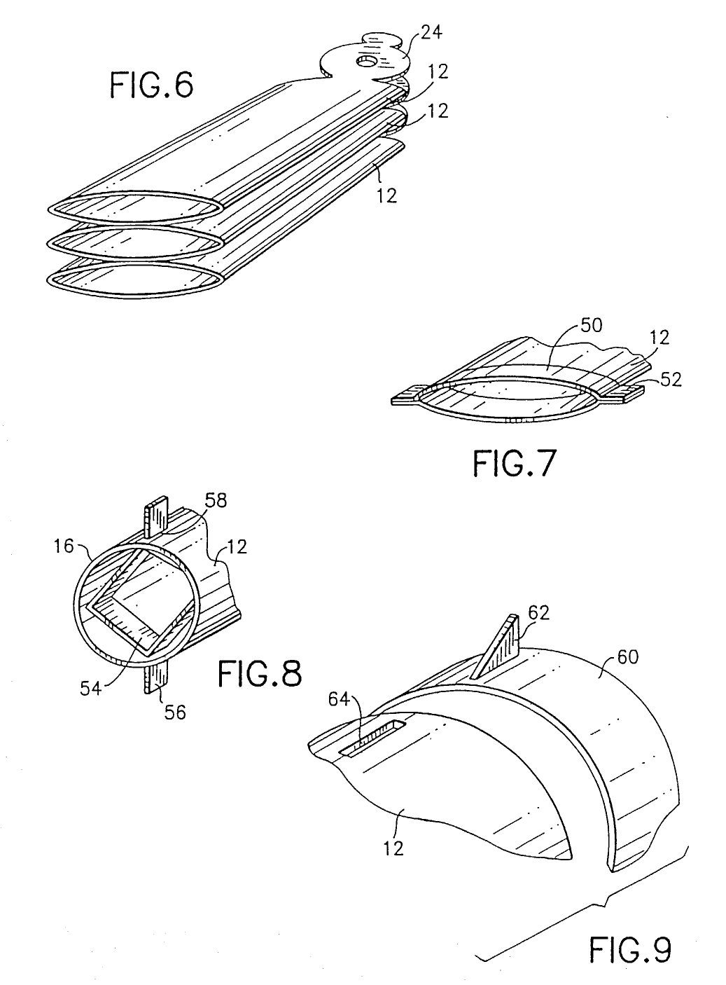

Referring now to FIG. 6, a plurality of bodies 12 in flat configuration are shown stacked one on top of the other. This configuration allows for numerous bodies to be shipped and stored in a space-efficient manner. The plastic securement members may be separately packed and shipped and stored along with the flat stacked bodies. When it is desirous to use needle holder assembly of the present invention, a flat blank 22 may be taken from storage and assembled as shown in FIGS. 3-5. Thereafter, the securement member 20 may be applied to flap 24 and secured thereto.

The needle holder 10 of the present invention may be used in conventional fashion. In particular, the needle holder 10 may be assembled as noted and the securement member 20 attached thereto. A separate needle assembly (not shown), typically a double-ended needle as is well known in phlebotomy procedures, is inserted through aperture 44 of the securement member 20 and secured thereto, such as through threaded engagement therewith. In this manner, the needle assembly extends within the interior 32 of the needle holder 10 through opening 26, and is secured to needle holder 10 within aperture 44. The needle can then be inserted into a blood vessel of a patient. After insertion, an evacuated sampling tube (not shown) is inserted within the open end 16 of body 12 and into the interior portion thereof. A piercable closure on the sampling tube can then be punctured by a non-patient puncture tip of the double-ended needle assembly extending through aperture 44, and a blood sample is automatically drawn into the sampling tube due to the negative pressure therein, as is known in the art.

After use, the needle holder assembly formed in accordance with the present invention may be easily discarded. It is further contemplated that needle holder 10 can be flattened after use by crushing the wall 14 of body 12 into a flattened configuration while maintaining the needle attached to the holder 10 through securement member 20. In this manner, the needle holder 10 can be easily discarded in a reduced profile configuration, thereby taking up less space in a waste container.

Referring to FIGS. 7-9, further embodiments of the present invention are shown.

FIG. 7 shows a separately applied end ring 50 which may be attached around the outside of wall 14 adjacent open end 16. End ring 50, which may be separately formed of cardboard or other suitable materials such as plastic, provides structural rigidity to the body 12 and also may provide for extending flanges 52 which help grip the needle holder assembly during use.

Additionally as shown in FIG. 8, a further end support 54 is shown. End support 54, which may also be formed of rigid cardboard or other suitable material such as plastic, may be generally rectangular in shape and may be expanded from a flat configuration. End member 54 also provides structural rigidity to the expanded body 12. The embodiment shown in FIG. 8 provides flanges 56 which extend through diametrically opposed slits 58 formed in wall 14 adjacent open end 16. End support 54 should include an opening therethrough which is sufficiently sized to accommodate a sampling tube (not shown) therethrough for insertion of the sampling tube within the interior portion of the expanded body 12 during use. It is further contemplated that such an opening be sized to support such a sampling tube when inserted within expanded body 12 during use. As such, end support 54 acts not only to provide structural rigidity to the expanded body 12, but also supports and properly centers a sampling tube within the interior of the expanded body 12 to provide for proper alignment during a sampling procedure.

Furthermore, rigid support ring 60 shown in FIG. 9 may be provided. Support ring 60 may also be formed of rigid cardboard or suitable plastic. Support ring 60 is inserted into the expanded open end 16 of body 12. In that regard, ring 60 includes tapered flanges 62 at diametrically opposite locations thereof which can be slideably inserted into diametrically opposed slits 64 formed adjacent to open end 16 of body 12.

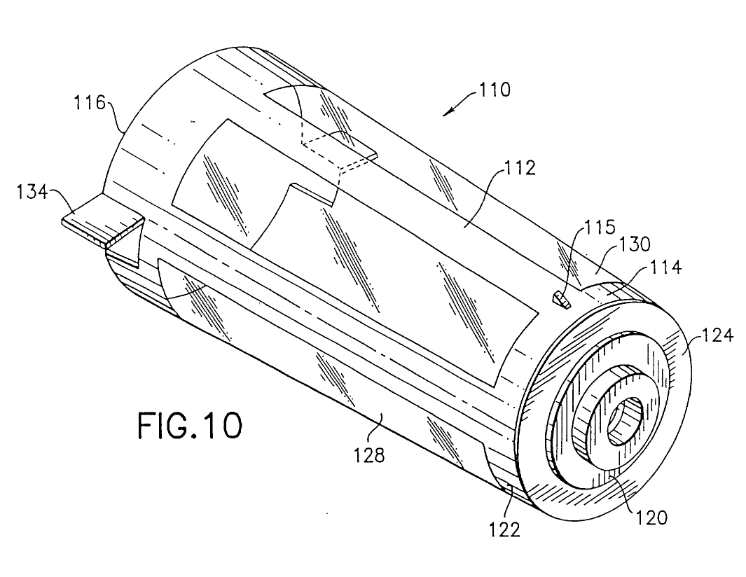

A further embodiment of the needle holder assembly of the present invention is shown in FIG. 10. In this embodiment, needle holder assembly 110 is formed from a flat blank 122 including a transparent viewing windows 128 formed therein. Upon expansion of the body 112 into a generally tubular cylindrical configuration, the viewing windows 128 provide the ability circumferentially view the interior thereof. The viewing windows 128 may include transparent plastic material 130 thereover. The embodiment of FIG. 10 further shows an alternate arrangement for flanges 134 which may be folded outwardly from diametrically opposite sides body 112 adjacent open end 116. Additionally, a further arrangement for securing flap 124 to body 112 is shown in FIG. 10 where a tab 115 extends through a slot in wall 114 of body 112 adjacent end 114. A needle securement member 120 may be similarly applied to body 112.

The embodiment of FIG. 10 is particularly well suited in embodiments in which the blank forming the needle holder is formed of an opaque material such as cardboard or paper. By providing the viewing window, at least a portion of the tubular wall will be transparent. It is noted that a transparent or translucent material such as a transparent resiliently flexible plastic may also be used as the entire material for the blank which forms the needle holder.

Moreover, it is recognized in the industry that needle holders are oftentimes used repeatedly for blood sampling procedures due to their durability. In particular, conventional plastic needle holders are typically very durable and resistant to collapsing or crushing. As such, it is common in the industry to re-use such conventional needle holders for repeated procedures on a plurality of patients. Even if errant blood or other body fluids are present on the walls of such conventional needle holders, it can be easily wiped off due to the rigid nature of the body of the needle holder. Such re-use, however, increases the risk of contamination and exposure to blood borne pathogens, and is therefore discouraged. In embodiments of the present invention in which the body of the needle holder is formed of a material such as cardboard or paper, any errant blood or body fluids would likely stain the wall of the body or may decrease the integrity of the wall of the needle holder due to moisture from the blood or body fluids. Any attempt to wipe the cardboard or paper walls of the needle holder would likely spread the stain of the blood or body fluids, and may further weaken the structure. As such, any errant blood or body fluids would provide evidence of re-use, and repeated use would be discouraged due to the staining or weakening of the walls from the blood or body fluids. It is also contemplated that such evidence of re-use through errant blood or body fluids may also be apparent with certain plastic materials useful in the collapsible needle holder of the present invention, and is not specifically limited to embodiments where the body is constructed of cardboard or paper.

Various other changes and modifications may be effected therein by one skilled in the art without departing from the scope and spirit of the invention, and is intended to claim all such changes and modifications as fall within the scope of the invention.

What is claimed:

1. A needle holder comprising:
an elongate body having a tubular wall and opposed ends, said tubular wall being radially expandable from a flat configuration to an expanded configuration defining openings at said ends; and
a flap extending from one of said ends and positionable across said opening at said one end to close said opening, said flap capable of supporting a needle assembly thereat.

2. A needle holder of claim 1, wherein said flap includes an extending tab, said tab being lockingly engageable with said tubular wall adjacent said one end.

3. A needle holder of claim 2, wherein said tubular wall includes a recess at said one end for lockingly accommodating said tab of said flap.

4. A needle holder of claim 1, wherein said tubular wall includes a pair of finger grip flanges extendable therefrom adjacent said other end.

5. A needle holder of claim 1, further including:
a needle securement member attachable to said flap.

6. A needle holder of claim 5, wherein said needle securement member includes a base portion supportable over said flap and a centrally located extending annular neck for supporting said needle assembly therethrough.

7. A needle holder of claim 1, wherein said body and said flap are integrally formed.

8. A needle holder of claim 1, wherein said body and flap are formed of a material selected from cardboard, paper and plastic.

9. A needle holder of claim 8, wherein at least a portion of said tubular wall includes a transparent material.

10. A needle holder of claim 8, wherein said tubular wall includes a viewing window.

11. A needle holder of claim 8, wherein said needle securement member is formed of plastic.

12. A needle holder of claim 1, further including a flange insert attachable to the other end of said body.

13. A needle holder of claim 12, wherein said flange insert includes a pair of diametrically opposing extending finger grip flanges extending therefrom.

14. A needle holder of claim 13, wherein said flange insert is attached within said body with finger grip flanges extending through the tubular wall.

15. A needle holder of claim 12, wherein said flange insert supports said wall in said expanded configuration.

16. A needle holder of claim 12, wherein said flange insert is formed of plastic.

17. A needle holder of claim 12, wherein said flange insert includes an opening adapted for centering a sampling tube within said body.

18. A needle holder comprising:
an elongate tubular wall capable of defining a cylindrical or conical tubular shape, said body having opposed opened ends, said tubular wall being radially expandable from a first shape of non-annular tubular cross-section to a second shape of annular tubular cross-section; and
a flap extending from one of said ends and positionable across at least one of said openings, said flap capable of supporting a needle assembly thereat.

19. The needle holder of claim 18, wherein said tubular wall comprises at least one crease.

20. The needle holder of claim 19, wherein said needle holder comprises an axis defined by said tubular wall in said second shape of annular cross-section, said at least one crease being coplanar with said axis.
